(19) United States
(12) Patent Application Publication
(10) Pub. No.: US 2014/0228701 A1
(43) Pub. Date: Aug. 14, 2014

(54) Brain-Computer Interface Anonymizer

(71) Applicant: University of Washington Through its Center for Commercialization, Seattle, WA (US)

(72) Inventors: Howard Jay Chizeck, Mercer Island, WA (US); Tamara Bonaci, Redmond, WA (US)

(21) Appl. No.: 14/174,818

(22) Filed: Feb. 6, 2014

Related U.S. Application Data
(60) Provisional application No. 61/763,339, filed on Feb. 11, 2013.

(51) Int. Cl. A61B 5/04; A61B 5/0478

(52) U.S. Cl. CPC A61B 5/04012; A61B 5/0478
USPC 600/544

(57) ABSTRACT

Methods and apparatus for using are provided for anonymizing neural signals of a brain-computer interface (BCI). A BCI can receive a plurality of brain neural signals. The plurality of brain neural signals can be based on electrical activity of a brain of a user and can include signals related to a BCI-enabled application. The BCI can determine features of the plurality of brain neural signals related to the BCI-enabled application. A BCI anonymizer of the BCI can generate anonymized neural signals by at least filtering the one or more features to remove privacy-sensitive information. The BCI can generate one or more application commands for the BCI-enabled application from the anonymized neural signals. The BCI can send the one or more application commands.

### CROSS-REFERENCE TO RELATED APPLICATIONS

The present application claims priority to U.S. Provisional Patent Application No. 61/763,339, entitled “Brain-Computer Interface Anonymizer”, filed Feb. 11, 2013, which is entirely incorporated by reference herein for all purposes.

### STATEMENT OF GOVERNMENT RIGHTS

This invention was made with government support under EEC-1028725 awarded by National Science Foundation (NSF). The government has certain rights in the invention.

### BACKGROUND

Unless otherwise indicated herein, the materials described in this section are not prior art to the claims in this application and are not admitted to be prior art by inclusion in this section.

A Brain-Computer Interface (BCI) is a communication system between the brain and the external environment. In this system, messages between an individual and an external world do not pass through the brain's normal pathways of peripheral nerves and muscles. Instead, messages are typically encoded in electrophysiological signals. Brain-computer interfaces can be classified as invasive, or involving implantation of devices; e.g., electrodes into the brain, partially invasive, or involving implantation of devices into a skull surrounding the brain, and non-invasive, or involving use of devices that can be removed; i.e., no devices are implanted into the brain or the skull.

The initial motivation for the development of brain-computer interfaces came from the growing recognition of the needs of people with disabilities, and of potential benefits brain-computer interfaces might offer. Brain-computer interfaces were first developed for assistance, augmentation and repair of cognitive and sensorimotor capabilities of people with severe neuromuscular disorders, such as spinal cord injuries or amyotrophic lateral sclerosis. More recently, however, BCIs have had a surge in popularity for non-medical uses, such as gaming, entertainment and marketing. Commonly supported applications of BCIs include (i) accessibility tools, such as mind-controlled computer inputs, such as a mouse or a keyboard, (ii) “serious games”, i.e., games with purpose other than pure entertainment, such as attention and memory training, and (iii) “non-serious” games for pure entertainment. Other applications are possible as well.

Most non-invasive brain-computer interfaces are based on electroencephalography (EEG), which involves directly measuring electrical potentials produced by neural synaptic activities from the brain. While EEG can be susceptible to noise and signal distortion, EEG signals are easily measurable and they have good temporal resolution. EEG-based BCIs are relatively popular for these reasons as well as their relatively low cost and low risk. Other brain-computer interfaces can use electrocorticography (ECoG) electrodes or electromyography (EMG) electrodes to obtain signals from the brain.

Event-Related Potentials (ERP) can be neurophysiological phenomena measured by EEG. An ERP is defined as a brain response to a direct cognitive, sensory or motor stimulus, and it is typically observed as a pattern of signal changes after the external stimulus. An ERP waveform consists of several positive and negative voltage peaks related to the set of underlying components. A sum of these components is caused by the “higher” brain processes, involving memory, attention or expectation.

Different ERP components can be used to infer things about a person's personality, memory and preferences. For example, data about a P300 ERP component has been used to recognize the person's name in a random sequence of personal names, to discriminate familiar from unfamiliar faces, and for lie detection. As another example, data about a N400 ERP component has been used to infer what the person was thinking about after he/she was primed on a specific set of words.

### SUMMARY

In one aspect, a method is provided. A brain-computer interface (BCI) receives a plurality of brain neural signals. The plurality of brain neural signals are based on electrical activity of a brain of a user and the plurality of brain neural signals include signals related to a BCI-enabled application. The brain-computer interface determines one or more features of the plurality of brain neural signals related to the BCI-enabled application. A BCI anonymizer of the brain-computer interface generates anonymized neural signals by at least filtering the one or more features to remove privacy-sensitive information. The brain-computer interface generates one or more application commands for the BCI-enabled application from the anonymized neural signals. The brain-computer interface sends the one or more application commands.

In another aspect, a brain-computer interface is provided. The brain-computer interface includes a signal acquisition component and a signal processing component. The signal acquisition component is configured to receive a plurality of brain neural signals based on electrical activity of a brain of a user. The plurality of brain neural signals includes signals related to a BCI-enabled application. The signal processing component includes a feature extraction component, a BCI anonymizer, and a decoding component. The feature extraction component is configured to determine one or more features of the plurality of brain neural signals related to the BCI-enabled application. The BCI anonymizer is configured to generate anonymized neural signals by at least filtering the one or more features to remove privacy-sensitive information. The decoding component is configured to generate one or more application commands for the BCI-enabled application from the anonymized neural signals.

In another aspect, an article of manufacture is provided. The article of manufacture includes a non-transitory tangible computer readable medium configured to store at least executable instructions, wherein the executable instructions, when executed by a processor of a brain-computer interface, cause the brain-computer interface to perform functions. The functions include: determining one or more features of a plurality of brain neural signals related to an BCI-enabled application; generating anonymized neural signals by at least filtering the one or more features to remove privacy-sensitive information; generating one or more application commands for the BCI-enabled application from the anonymized neural signals; and sending the one or more application commands from the brain-computer interface.

The herein-disclosed brain-computer interface provides at least the advantage of enabling a BCI user to control aspects of their privacy that might otherwise be obtained via the BCI. The brain-computer interface uses a BCI anonymizer to prevent releasing information. In some cases, the control of information is provided on a per-component basis, where an example component is an event-related potential (ERP) component. The BCI anonymizer both protects brain-computer interface users and aids adoption of the use of BCI's by minimizing user risks associated with brain-computer interfaces.

### BRIEF DESCRIPTION OF THE DRAWINGS

FIG. 1A illustrates a system where a user is using a brain-computer interface to communicate with a computing device, in accordance with an embodiment;

FIG. 1B illustrates another system where a user is using a brain-computer interface to communicate with a computing device, in accordance with an embodiment;

FIG. 1C illustrates a system where a user is using a brain-computer interface to communicate with a robotic device, in accordance with an embodiment;

FIG. 2A shows an example EEG graph, in accordance with an embodiment;

FIG. 2B shows a graph of simulated voltages over time indicating various ERPs, in accordance with an embodiment;

FIG. 2C shows a graph of example P300 ERP potentials plotted with respect to time, in accordance with an embodiment;

FIG. 3 is a block diagram of a system for transforming brain neural signals to application-specific operations, in accordance with an embodiment;

FIG. 4 is a flowchart of a calibration method, in accordance with an embodiment;

FIG. 5A is a block diagram of an example computing network, in accordance with an embodiment;

FIG. 5B is a block diagram of an example computing device, in accordance with an embodiment; and

FIG. 6 is a flow chart of an example method, in accordance with an embodiment.

### DETAILED DESCRIPTION

A person using a brain-computer interface can put their privacy at risk. Research has been directed to potential benefits of using brain neural signals data for user identification; e.g., selecting a user's identity out of a set of identities, and authentication; verification that a claimed identity is valid, based on the observation that brain neural signals of each individual are unique. EEG signals, such as those signals captured by brain-computer interface, have shown to be particularly useful for these applications. That is, the EEG signals captured from a user of a brain-computer interface could be used to identify the user and/or authenticate the user's identity, perhaps even without the user's consent.

Scholarly papers have addressed privacy concerns related with brain computer interfaces. In 2005, The Committee on Science and Law considered possible legal implications of neural engineering, in particular on the use of neural imaging in non-medical research. The committee recognized neuromarketing, defined as the field of marketing research that studies consumers' sensorimotor, cognitive, and affective response to marketing stimuli and brain fingerprinting, defined as a technique that purports to determine the truth by detecting information stored in the brain, as emerging non-medical areas using neural imaging data. In a 2011 paper, the author presented several examples, showing how modern neuroscience expected to facilitate evidence collection during criminal investigation. The presented examples indicate the traditional border between testimonial and physical evidence becomes blurry when applied to data collected by neural engineering techniques.

Recent experimental results show that EEG signals can be used to extract private information about a user. With sufficient computational power, this information can be exploited to determine about privacy-sensitive information, such as memory, intentions, conscious and unconscious interests, and emotional reactions of a brain-computer interface user. For example, “brain spyware” for brain-computer interfaces has been developed that uses ERP components, particularly the P300 ERP component, to exploit privacy-sensitive information to obtain data about a person's finances, biographical details, and recognized colleagues.

Some examples can illustrate some uses, both legitimate and malicious, of privacy-sensitive information obtainable from brain-computer interfaces:
Access to an individual's memories and emotional responses might be used by police enforcement and government agencies during criminal investigation, as well as for crime and terrorism prevention.
BCI-recorded brain neural signals can be used in a variety of entertainment and relaxation applications. A person's emotional response and satisfaction/dissatisfaction level may, for example, be used to provide better (more accurate) music and/or movie recommendations. Similarly, information about a person's activity and anxiety levels may be used to tailor a more personalized training routine or a relaxation session.
Privacy-sensitive information can be used to target advertisements, as an advertiser can have a real-time access to a person's level of interest, satisfaction, or frustration with the presented material.
Privacy-sensitive information, such as extracted information about a person's memories, prejudices, beliefs or possible disorders, can be used to manipulate or coerce a person into doing otherwise unthinkable by the person.
Privacy-sensitive information could also be used to cause physical or emotional pain to a person. Examples of such actions have already been observed; e.g., flashing animations were placed on epilepsy support webpages, eliciting seizures in some patients with photosensitive epilepsy.

A BCI anonymizer can be used to address these privacy concerns. The BCI anonymizer includes software, and perhaps hardware, for decomposing brain neural signals in into a collection of characteristic signal components. The BCI anonymizer can extract information corresponding to intended BCI commands and remove private side-channel information from these signal components. Then, the BCI anonymizer can provide a suitably configured application with only the signal components related to the BCI commands for the application. That is, the BCI anonymizer can provide BCI-command-related information to an application without providing additional privacy-sensitive information.

The BCI anonymizer can process brain neural signals in real time and provide only signal components required by the application, rather than providing the entire brain neural signal. This real time approach mitigates privacy attacks that might occur during storage, transmission, or data manipulation by a BCI application. For example, if complete brain neural signals were transmitted, an eavesdropper can intercept the transmission, save the brain neural signals, and decompose the saved brain neural signals to obtain privacy-sensitive information. Thus, real time operation of the BCI anonymizer can significantly decrease the risk to privacy-sensitive information since complete brain neural signals are neither stored by nor transmitted from the BCI anonymizer.

While BCI-command-related information provides some information about the state of mind of the user, the BCI anonymizer reduces, if not eliminates, risk to privacy-sensitive information by removing side-channel information about additional ERP components not related to the BCI commands. This side channel information can be used to increase the success rate of extracting privacy-sensitive information. In some embodiments, the BCI anonymizer can provide information only about specifically authorized ERP components and/or filter out information about other ERP components. For example, the BCI anonymizer can be configured to provide (a) information about specific ERP component(s) that may be tied to specific applications; e.g., information about the ERN (error-related negativity) component to a document-management program or information about the N100 component to a game, (b) exclude providing information specific ERP component(s); e.g., exclude information about the P300 component, and/or (c) provide information about ERP component(s) after specific authorization by the user.

The BCI anonymizer protects brain-computer interface users from unintentionally providing privacy-sensitive information. The BCI anonymizer acts in real-time to provide requested ERP component information without sharing or storing complete brain neural signals. By providing this protection, the BCI anonymizer can ensure private thoughts remain private even during use of the brain-computer interface. This assurance can reduce the risks of using brain-computer interfaces, and speed adoption and use of the brain-computer interface.

Example Systems Utilizing BCI Anonymizers

A brain-computer interface can be used to decode (or translate) electrophysiological signals, reflecting activity of central nervous system, into a user's intended messages that act on the external world. A brain-computer interface can act as a communication system, with inputs about a user's neural activity, outputs related to external world commands, and components decoding inputs to outputs. A brain-computer interface can include electrodes, as well as signal acquisition and signal processing components for decoding inputs to outputs. Brain-computer interfaces often have relatively low transmission rates; e.g., a transmission rate between 10 and 25 bits/minute.

As indicated above, brain-computer interfaces can be attacked to obtain information, such as privacy-sensitive information. For example, consider an attacker who uses brain-computer interfaces, such as non-invasive brain-computer interfaces intended for consumer use, for extracting privacy-sensitive information.

Generally speaking, two types of attackers can use brain-computer interfaces to extract privacy-sensitive information, where the two types are based on the way an attacker analyzes recorded brain neural signals. The first type of attacker extracts users' private information by hijacking the legitimate components of the brain-computer interface; e.g., exploiting legitimate outputs of the brain-computer interface for the attacker's own purposes. The second type of an attacker extracts users' private information by adding or replacing legitimate brain-computer interface components. For example, the second type of attacker can implement additional feature extraction and/or decoding algorithms, and either replaces or supplements the existing BCI components with the additional malicious code. The difference between the two attacker types is only in the structure of a malicious component—the first type of attacker attacks outputs produced by the brain-computer interface, while the second type of attacker attacks the brain-computer interface components.

The attacker can interact with users by presenting them with specific sets of stimuli, and recording their responses to the presented stimuli. Some example techniques that the attacker can present stimuli to users include:
Oddball paradigm—a technique where users are asked to react to specific stimuli, referred to as target stimuli, hidden as rare occurrences in a sequence of more common, non-target stimuli;
Guilty knowledge test—a technique based on the hypothesis that a familiar stimulus evokes a different response when viewed in the context of similar, but unfamiliar items; and
Priming—a technique that uses an implicit memory effect where one or more stimuli may influence a person's response to a later stimulus or stimuli.
   Other techniques that the attacker can present stimuli to users are possible as well.

The attacker can use these techniques, and perhaps others, to facilitate extraction of private information. In addition, an attacker can present malicious stimuli in an overt (conscious) fashion, as well as in a subliminal (unconscious) way, with subliminal stimulation defined as the process of affecting people by visual or audio stimuli of which they are completely unaware; e.g., the attacker can reduce a stimulus intensity or duration below the required level of conscious awareness by the user.

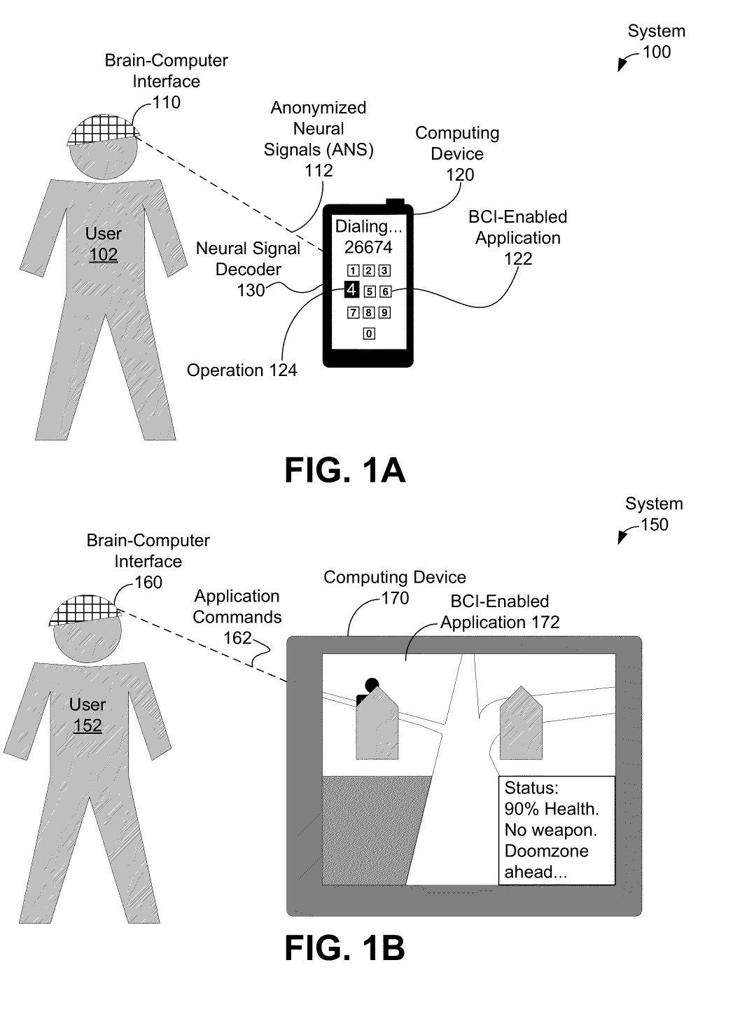

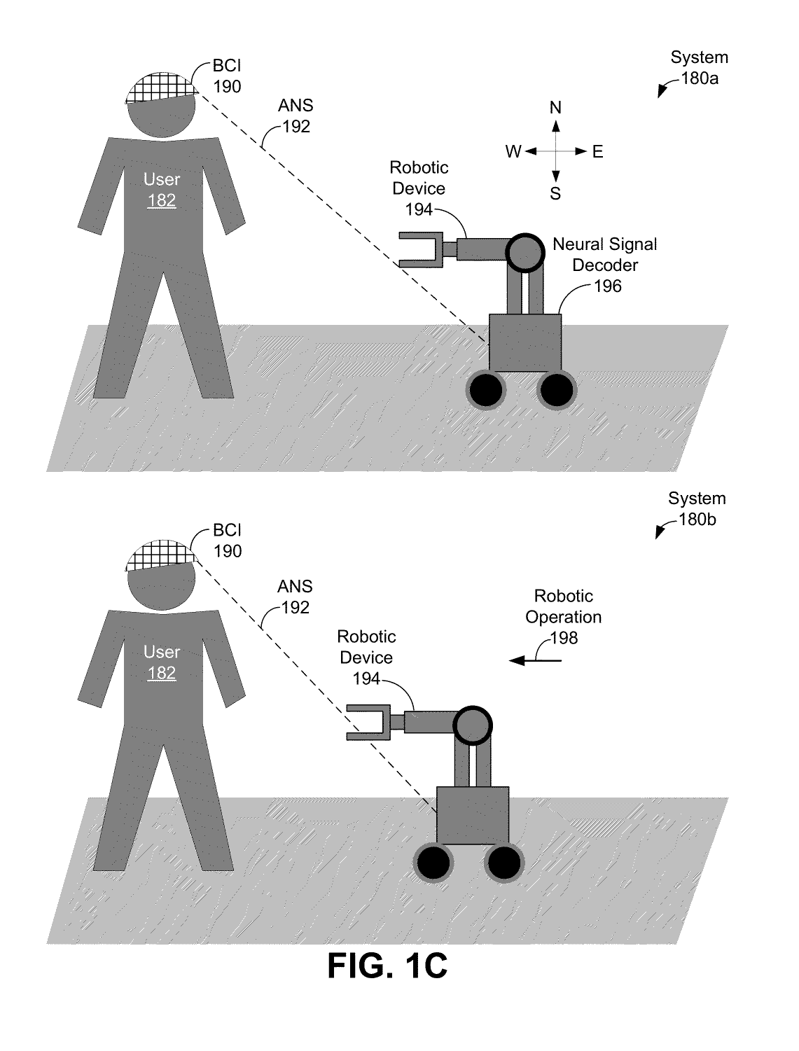

In the example systems shown in FIGS. 1A-1C, each brain-computer interface further includes a BCI anonymizer. A BCI anonymizer can thwart attackers such as discussed above, and so enhance neural privacy and security. The BCI anonymizer can pre-process brain neural signals before they are stored and transmitted to only communicate information related to intended BCI commands. The BCI anonymizer can prevent unintended information leakage by operating in real-time without transmitting or storing raw brain neural signals or signal components that are not explicitly needed for the purpose of legitimate application-related data; e.g., commands to the application generated by the brain-computer interface.

FIG. 1A illustrates system 100 with user 102 using brain-computer interface 110 to communicate with computing device 120. FIG. 1A shows that user 102 is in the process of making a phone call using BCI-enabled application 122 of computing device 120. User 102 has dialed the digits “26674” as displayed by application 122, which also indicates operation 124 of dialing the digit “4”.

User 102 can generate analog brain neural signals that can be captured by brain-computer interface 110. Brain-computer interface 110 can include electrodes that capture brain neural signals generated by a brain of user 102, as shown in FIG. 1A. Brain-computer interface 110 can convert analog brain neural signals obtained via the included electrodes to digital brain neural signals, anonymize the digital brain neural signals using the BCI anonymizer of brain-computer interface 110, and send anonymized neural signals (ANS) 112 to computing device 120.

Anonymized neural signals 112 can be correlated to information an application of computing device 120, such as BCI-enabled application 122. BCI-enabled application 122 can include, or communicate with, neural signal decoder 130. Neural signal decoder 130 can decode anonymized neural signals 112 into commands recognizable by BCI-enabled application 122.

For example, the commands can correspond to application operations, such as touching a specific digit on a keypad, such as keypad displayed by BCI-enabled application 122 and shown in FIG. 1A. FIG. 1A shows example operation 124 of touching the number “4” on the keypad. In response to operation 124, BCI-enabled application 122 can act as if user 102 touched the number “4” on the keypad with a finger. That is, BCI-enabled application 122 can update the display of the keypad to show the touch of the number “4” and has added the digit 4 to digits dialed as shown on computing device 120.

FIG. 1B illustrates system 150 with user 152 using brain-computer interface 160 to communicate with computing device 170. FIG. 1B shows that user 152 is playing a game using application 172 of computing device 170. User 152 can generate analog brain neural signals using brain-computer interface 160. As with brain-computer interface 110, brain-computer interface 160 can include electrodes that can acquire brain neural signals from the brain of a user, such as user 152.

Brain-computer interface 160 can convert analog brain neural signals obtained via the included electrodes to digital brain neural signals, anonymize the digital brain neural signals using the BCI anonymizer of brain-computer interface 160, decode the anonymized brain neural signals to application commands 162, and send application commands 162 to computing device 170. Computing device 170 can provide application commands 162 to application 172 so that user 152 can play the game provided by application 172.

Users can use brain-computer interfaces to communicate with devices other than computing devices 120 and 170, such as, but not limited to, remotely-controllable devices and robotic devices.

FIG. 1C illustrates systems 180a, 180b with user 182 using brain-computer interface 190 to communicate with robotic device 194. System 180a, shown in the upper portion of the sheet for FIG. 1C, shows user 182 using brain-computer interface 190 to communicate with robotic device 194. Brain-computer interface 190 can convert analog brain neural signals obtained via the included electrodes to digital brain neural signals, anonymize the digital brain neural signals using the BCI anonymizer of brain-computer interface 190, and send anonymized neural signals 192 to robotic device 194. Anonymized neural signals 192 can be correlated to control information for robotic device 194 using neural signal decoder 196. That is, neural signal decoder 196 can decode anonymized neural signals 192 into robotic operations recognizable by robotic device 194.

The robotic operations can include, such as, but are not limited to: (1) an operation to moving a robot in a direction; e.g., left, right, forward, backward, up, down, north, south, east, west; (2) an operation to rotate the robot in a direction, (3) moving an end effector; e.g., an arm of the robot in a direction; (4) rotating an end effector in a direction, (5) operating the end effector; e.g., opening a hand, closing a hand, rotating an end effector, (6) power up/down the robot, (7) and provide maintenance/status information about the robot; e.g., information to diagnose and repair the robot, battery/power level information.

In the example shown in FIG. 1C, user 182 uses brain-computer interface 190 to provide anonymized neural signals 192 to robotic device 194 corresponding to a robotic operation to move robotic device 194 closer to user 182; e.g., move in a westward direction. System 180b shows a configuration after robotic device 194 has carried out robotic operation 198 to move westward toward user 182.

Brain-Computer Interfaces with BCI Anonymizers

A brain-computer interface with a BCI anonymizer can also include electrodes to capture electrical indicia of brain neural signals as well as signal acquisition and signal processing components for decoding (translating) inputs, such as the captured electrical indicia of brain neural signals, to outputs such as anonymized neural signals, as indicated with respect to FIGS. 1A and 1C and/or application commands, as indicated with respect to FIG. 1B.

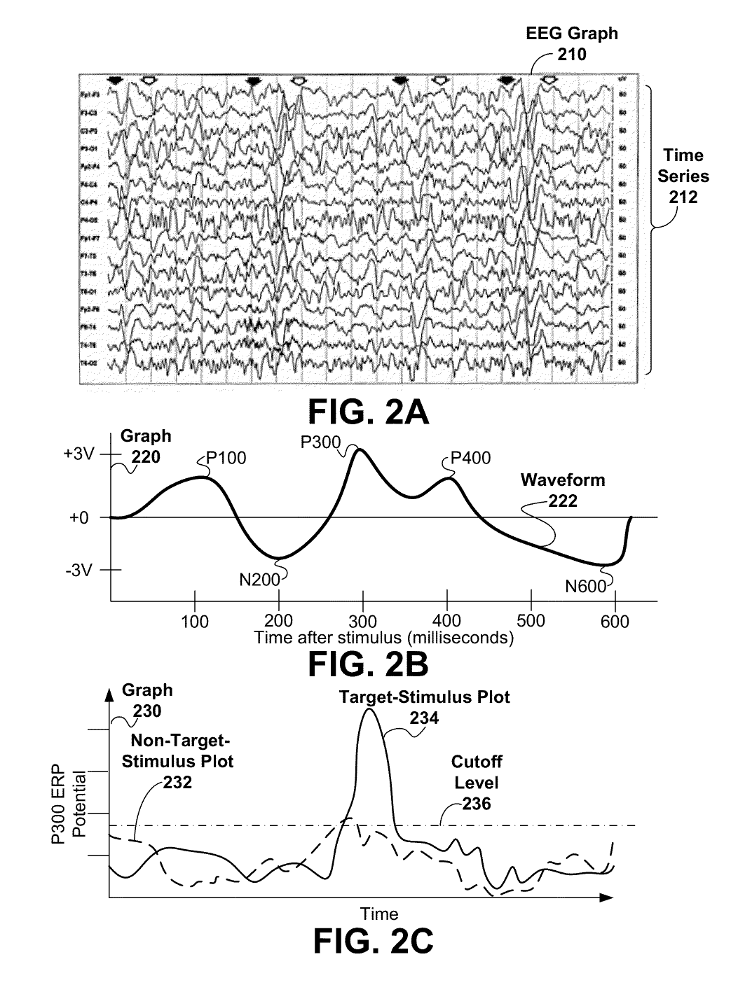

FIG. 2A shows example EEG graph 210. During calibration and operation of the brain-computer interface, brain neural signals can be collected that were generated in response to various stimuli. The brain neural signals can be collected as a number of time series; e.g., using a 16-channel EEG-based brain-computer interface, 16 different time series can be collected such as shown as time series 212 of FIG. 2A. Each time series can represent voltages, or other electrical characteristics, over time, where the voltages can be generated by neurons of a human brain as brain neural signals, and where the brain neural signals can be collected by electrode(s) of the brain-computer interface located over specific portion(s) of the human brain.

The signal acquisition component can prepare input brain neural signals for signal processing. For example, the signal acquisition component can amplify and/or otherwise condition input analog brain neural signals, convert the analog signals to digital, and perhaps preprocess either the analog brain neural signals or the digital brain neural signals for signal processing.

For example, acquired time series can be preprocessed. Preprocessing a time series can include adjusting the time series based on a reference value. Other types of preprocessing time series, analog brain neural signals, and/or digital brain neural signals are possible as well. In some embodiments, preprocessing occurs during signal acquisition. In other embodiments, preprocessing occurs during signal processing, while in still other embodiments, preprocessing occurs during both signal acquisition and signal processing.

In some scenarios; e.g., during calibration, the time series can represent repeated actions; e.g., EEG signals captured after a brain-computer interface user was presented with the above-mentioned sequence of stimuli, the EEG signals can be segmented into time-based trials, and the signals for multiple trials averaged. Multiple-trial averaging can be carried out by the signal acquisition component and/or the signal processing component.

The signal processing component can have a feature extraction component, a decoding component, and a BCI anonymizer. The brain-computer interface generally, and a BCI anonymizer of the brain-computer interface specifically, can be calibrated for a user of a brain-computer interface.

The time series can be filtered; e.g. to isolate ERP components. ERPs can be relatively-low frequency phenomena, e.g., less than 30 Hz. Generally, ERPs can have a frequency less than a predetermined number of occurrences per second, where the predetermined number can depend on a specific ERP. To isolate ERPs, high-frequency noise can be filtered out from time series by a low-pass filter. The specific low-pass filter can be determined by the parameters of the ERP component; e.g., N100, P300, N400, P600 and ERN ERP components. Other filters can be applied as well; e.g., filters to remove eye-blink and/or other movement data that can obscure ERP components. Filtering can be performed during signal acquisition and/or during feature extraction.

The feature extraction component can process recorded brain neural signals in order to extract signal features that are assumed to reflect specific aspects of a user's current mental state, such as ERP components. FIG. 2B shows graph 220 of simulated voltage data from an EEG channel graphed over time indicating various ERPs. Graph 220 simulates reaction observed by an electrode of a brain-computer interface after a baseline time of 0 milliseconds (ms) when a stimulus was generated. Graph 220 includes waveform 222 with peaks of positive voltage at approximately 100 ms, 300 ms, and 400 ms, as well as valleys of negative voltage at approximately 200 ms and 600 ms. Each of these peaks and valleys can represent an ERP.

Naming of ERPs can include a P for a positive (peak) voltage or N for a negative (valley) voltage and a value indicating an approximate number of ms after a stimulus; e.g., an N100 ERP would represent a negative voltage about 100 ms after the stimulus. Using this nomenclature, the positive peaks of waveform 222 of FIG. 2B at approximately 100 ms, 300 ms, and 400 ms can each respectively represent a P100 ERP, a P300 ERP, and a P400 ERP and the valleys of waveform 222 at approximately 200 ms and 600 ms can each respectively represent a N200 ERP and N600 ERP.

A number of ERP components have been discovered and used in neuroscience. ERP components can be broadly be classified into: (a) visual sensory responses, (b) auditory sensory responses, (c) somatosensory, olfactory and gustatory responses, (d) language-related ERP components, (e) error detection, and (f) response-related ERP components. Among these ERP components, certain ERP components can be considered to relate to privacy-sensitive information, including but not limited to an N100 ERP component, a P300 ERP component, a N400 ERP component, a P600 ERP component, and error-detection ERN ERP component.

Research indicates that these ERP components may reflect the following reactions and processes:
N100—a reaction to any unpredictable stimulus,
P300—processes involving stimulus evaluation or categorization,
N400—a reaction to a meaningful or potentially meaningful stimulus, including words, pictures, sounds, smells or faces,
P600—a reaction to hearing or reading a grammatical error, or other syntactic anomaly, and
ERN—processes occurring after an error is committed in multiple-choice tasks, even if a person is not explicitly aware of the error.

A large body of research has investigated how different ERP components can be used to infer information about a user's intent, cognitive and behavioral processes, as well as about his/her affective and emotional states. For example, a P300 ERP component, typically observed over the parietal cortex as a positive peak at about 300 milliseconds after a stimulus, is typically elicited as a response to infrequent or particularly significant auditory, visual or somatosensory stimuli, when interspersed with frequent or routine stimuli. One of the important advantages of a P300-based brain-computer interface is the fact that the P300 is typical, or naive, response to a desired choice, thus requiring no initial user training. One application of the P300 response is a spelling application, the P300 Speller, proposed and developed by Farwell et al. in 1988. More recently, the P300 response has been used to recognize a subjects name in a random sequence of personal names, to discriminate familiar from unfamiliar faces, and for lie detection.

Another well-investigated component is the N400 response, associated with semantic processing. The N400 has recently been used to infer what a person was thinking about, after he/she was primed on a specific set of words. This ERP component has also been linked to the concept of priming, an observed improvement in performance in perceptual and cognitive tasks, caused by previous, related experience. In addition, the N400 and the concept of priming have had important role in subliminal stimuli research.

Similarly, the P600 component has been used to make an inference about a user's sexual preferences, and the estimates of the anxiety level derived from the EEG signals has been used to draw conclusions about a person's religious beliefs.

In some embodiments, the feature extraction component can have a classifier to extract ERP components from the time series. For example, a classifier based on a logistic regression algorithm can be used. As another example, a classifier based on stepwise linear discriminant analysis; e.g., the BCI2000 P300 classifier. Other classifiers are possible as well.

FIG. 2C shows graph 230 of example P300 ERP potentials plotted with respect to time. Graph 230 shows plots of potentials of the P300 ERP component over time resulting after presentation of two separate stimuli. Non-target-stimulus plot 232 represents responses from a non-target stimulus; e.g., a stimulus not expected to be categorized, while target-stimulus plot 234 represents responses from a target stimulus. In particular, the non-target stimulus was an image of a face unfamiliar to the brain-computer interface use, while the target stimulus was an image of a well-known person's face.

Considering the example shown in FIG. 2C, the BCI anonymizer can extract reactions to target stimuli from only the P300 ERP components by using a high-pass filter that removes all P300 potentials below a cutoff level; e.g., cutoff level 236 as shown in FIG. 2C. The BCI anonymizer can be realized in hardware and/or software.

In some embodiments, the BCI anonymizer can use time-frequency signal processing algorithms for real time decomposition of brain neural signals into one or more functions. The BCI anonymizer can then construct anonymized brain neural signals by altering some or all of the one or more functions to protect user privacy. After altering the one or more functions, the BCI anonymizer can reconstruct the brain neural signals from the altered one or more functions. That is, the reconstructed brain neural signals can represent anonymized brain neural signals that contain less privacy-sensitive information than the input brain neural signals. Then, the anonymized brain neural signals can be provided to another component of a brain-computer interface and/or an application, such as a BCI-enabled application.

The decoding component can use decoding algorithms to take the signal features as inputs, which may be represented as feature vectors. The decoding algorithms can transform the signal features into application-specific commands. Depending on the application, many different decoding algorithms can be used by the brain-computer interface. For example, a decoding algorithm can adapt to: (1) individual user's signal features, (2) spontaneous variations in recorded signal quality, and (3) adaptive capacities of the brain (neural plasticity).

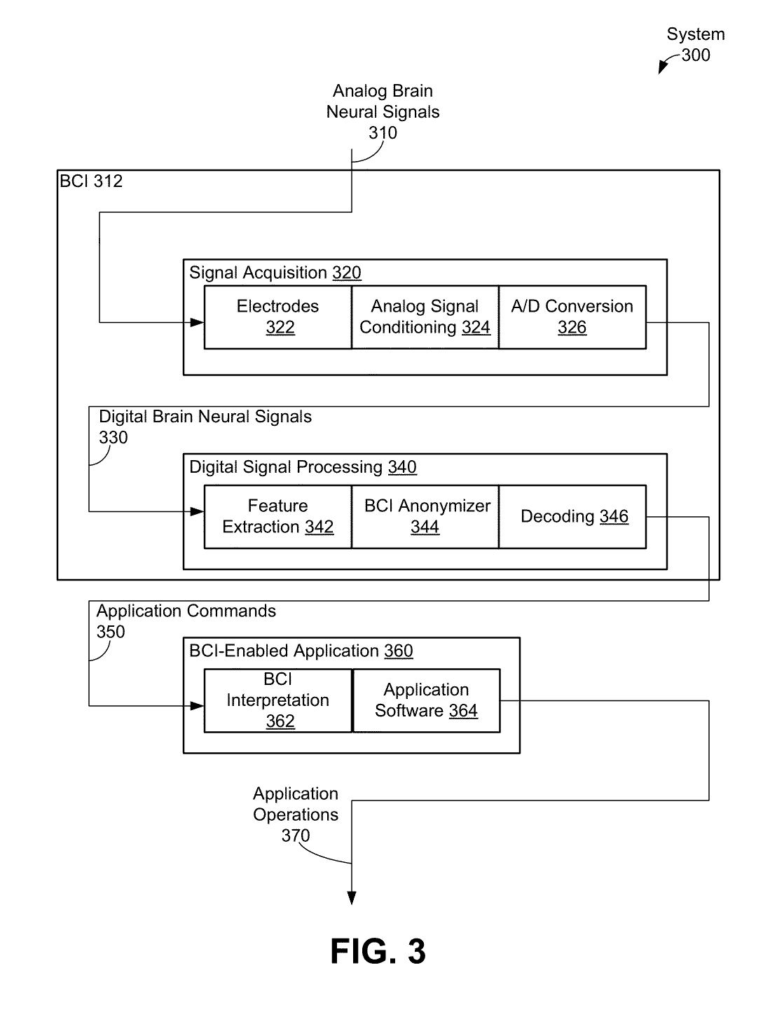

FIG. 3 is a block diagram of system 300 for transforming brain neural signals to application-specific operations. System 300 includes a brain-computer interface 312 and a BCI-enabled application 360. The brain-computer interface 312 includes signal acquisition component 320 and digital signal processing component 340.

Signal acquisition component 320 can receive analog brain neural signals 310 from a brain of a user, such as discussed above in the context of FIGS. 1A-1C, and generate digital brain neural signals 330 as an output. Signal acquisition component 320 can include electrodes 322, analog signal conditioning component 324, and analog/digital (A/D) conversion component 326. Electrodes 322 can obtain analog brain neural signals 310 from brain of a user and can include, but are not limited to, some or all of non-invasive electrodes, partially invasive electrodes, invasive electrodes, EEG electrodes, ECoG electrodes, EMG electrodes, dry electrodes, wet electrodes, wet gel electrodes, and conductive fabric patches.

Electrodes 322 can be configured to provide obtained analog brain neural signals to analog signal conditioning component 324. For example, the analog brain neural signals can be time series as discussed above in the context of FIGS. 1A-1C. Analog signal conditioning component 324 can filter, amplify, and/or otherwise modify the obtained analog brain neural signals to generate conditioned analog brain neural signals. Analog signal conditioning component 324 can include but is not limited to amplifiers, operational amplifiers, low-pass filters, band-pass filters, high-pass filters, anti-aliasing filters, other types of filters, and/or signal isolators.

In some embodiments, analog signal conditioning component 324 can receive multiple time series of data from the brain. These signals from the time series can be preprocessed by adjusting the signals based on a reference value. For example, let the values of n time series at a given time be SV1, SV2 . . . SVn, and let M, the mean signal value be

$$M = \frac{1}{n} {\sum }_{i = 1}^{n} SVi .$$

Then, each signal value can be adjusted by subtracting the mean signal value M from the signal value; e.g., for a given signal value j, 1≦j≦n, SVj=SVj−M. Other reference values can include a predetermined numerical value or a weighted average of the signal values. Other reference values and other types of preprocessing are possible as well. In some embodiments, the preprocessing is performed by digital signal processing component 340.

Analog signal conditioning component 324 can be configured to provide conditioned analog brain neural signals to analog/digital conversion component 326. Analog/digital conversion component 326 can sample conditioned analog brain neural signals at a sampling rate; e.g., 256 samples per second, 1000 samples per second. The obtained samples can represent voltage, current, or another quantity. A sample can be resolved into a number of levels; e.g., 16 different levels, 256 different levels. Then, digital data such as a bitstream of bits for each sample representing a level for the sample can be output as digital brain neural signals 330.

For example, if a current is sampled between 0.01 and 0.21 amperes and resolved into four levels, the four levels can correspond to current in level 0 of 0.01 to 0.05999 . . . amperes, level 1 of 0.06 to 0.10999 . . . amperes, level 2 of 0.11 to 0.15999 . . . amperes, and level 3 of 0.16 to 0.21 amperes. These four levels can be represented using two bits; e.g., bits 00 for level 0, bits 01 for level 1, bits 10 for level 2, and bits 11 for level 3.

As another example, suppose a conditioned analog brain neural signal has a voltage range from V1 volts to V2 volts, and the brain neural signal is sampled within the voltage range and resolved into sixteen levels. Then, analog/digital conversion component 326 can output each sample as four bits that represent the sixteen levels. Many other sampling rates, sampled quantities, and resolved number of levels are possible as well.

Digital signal processing component 340 can receive digital brain neural signals 330 from signal acquisition component 320 as inputs and generate application commands 350 as output(s). For example, digital signal processing component 340 can be part or all of one or more digital signal processors. In some embodiments, feature extraction component 342 and BCI anonymizer 344 can be a single component. In other embodiments, BCI anonymizer 344 and decoding component 346 can be a single component. In still other embodiments, decoding component 346 can be part of BCI-enabled application 360; in those embodiments, such as discussed above in the context of FIGS. 1A and 1C, brain-computer interface 312 can provide anonymized neural signals to BCI-enabled application 360 and the decoding component of the BCI-enabled application can decode the anonymized digital neural signals into application commands; e.g., commands equivalent to application commands 350.

Digital signal processing component 340 can include feature extraction component 342, BCI anonymizer 344, and decoding component 346. Feature extraction component 342 can receive conditioned digital brain neural signals as inputs and generate extracted features, such as ERP components, as outputs. In some embodiments, feature extraction component 342 can preprocess input time series that are in digital brain neural signals 330 by adjusting the time series based on a reference value, such as discussed above in the context of analog signal conditioning component 324.

To determine the features of the digital brain neural signals 330, feature extraction component 342 can perform operations, such as filtering, rectifying, averaging, transforming, and/or otherwise process, on digital brain neural signals 330. For example, feature extraction component 342 can use one or more high-pass filters, low-pass filters, band-pass filters, finite impulse response (FIRs), and/or other devices to filter out noise from digital brain neural signals unrelated to ERP components.

Then, feature extraction component 342 can have a classifier to extract features, such as ERP components, from noise-filtered digital brain neural signals 330. For example, a classifier based on a logistic regression algorithm can be used. As another example, a classifier based on stepwise linear discriminant analysis; e.g., the BCI2000 P300 classifier. In some embodiments, the classifier can be trained to extract multiple features; e.g., multiple ERP components, while in other embodiments, multiple classifiers can be used if multiple features are to be extracted; e.g., one classifier for N100 ERP components, one classifier for P300 ERP components, and so on.

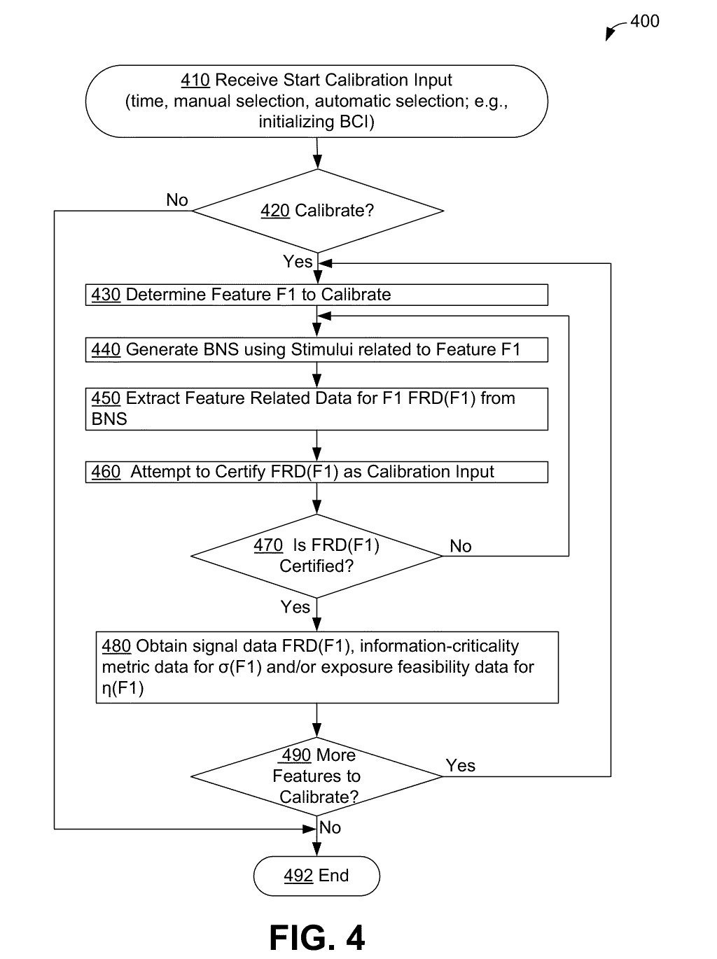

The extracted features can be provided to BCI anonymizer 344, which can filter information to determine an amount of privacy-sensitive information to be provided to BCI-enabled application 360. For example, BCI anonymizer 344 can determine an amount of privacy-sensitive information to be provided to BCI-enabled application 360 based on data about privacy-sensitive information related to a user of brain-computer interface 312, such as data about information-criticality metrics and relative reductions in entropy, and information about features utilized by BCI-enabled application 360. Information-criticality metrics and relative reductions in entropy are discussed below in more detail in the context of FIG. 4.

For example, suppose an ERP component E1 is designated by a user U1 as being unimportant to user privacy. Then, BCI anonymizer 344 can allow much, if not all information, about E1 to BCI-enabled application 360. As another example, suppose user U2 designates E1 as being very important to user privacy. Then, BCI anonymizer 344 can restrict information about E1 to BCI-enabled application 360, unless E1-related data can be legitimately used by BCI-enabled application 360. In cases where data about a feature is important to both user privacy and to an application, BCI anonymizer 344 can allow data about the feature to be used by BCI-enabled application 360 with specific authorization from the user. This authorization can be provided at the time that the user uses BCI-enabled application 360 or during calibration or another time; e.g., provide default authorization to allow data about the feature.

BCI anonymizer 344 can restrict information about non-target stimuli for an ERP component. For example, BCI anonymizer 344 can extract reactions to target stimuli by using a high-pass filter that removes all potentials below a cutoff level, under the assumption that data below the cutoff level relates to non-target stimuli, such as discussed above in the context of the P300 ERP component and FIG. 2C. The cutoff level can be based on importance values related to user privacy and to uses of BCI-enabled application 360 as well as signal-related aspects of the ERP component observed during calibration or other times.

However, an attacker could detect that anonymized neural signals about the feature are too “clean”; e.g., no data is presented for potentials below the cutoff level. Then, BCI anonymizer 344 can mix a clean signal with other signal(s) before transmission; e.g., a random signal whose potential is below the cutoff level, a recorded signal known not to carry any privacy-related information, a simulated signal of a non-target stimulus, and/or other signals partially or completely below the cutoff level.

In some embodiments, BCI anonymizer 344 can use time-frequency signal processing algorithms to decompose brain neural signals in real time into one or more functions. BCI anonymizer 344 can then construct anonymized brain neural signals by altering some or all of the one or more functions to protect user privacy. After altering the one or more functions, BCI anonymizer 344 can reconstruct the brain neural signals from the altered one or more functions. That is, the reconstructed brain neural signals can represent anonymized brain neural signals that contain less privacy-sensitive information than the input brain neural signals. Then, the anonymized brain neural signals can be provided to another component of a brain-computer interface; e.g., decoding component 346, and/or an application; e.g., BCI-enabled application 360.

In some embodiments, BCI anonymizer 344 can use time-frequency signal processing algorithms for real time decomposition of brain neural signals. For example, the BCI anonymizer can use Empirical Mode Decomposition (EMD) to perform nonlinear and non-stationary data signal processing. EMD is a flexible data driven method for decomposing a time series into multiple intrinsic mode functions (IMFs) where IMFs can take on a similar role as basis functions in other signal processing techniques.

In particular embodiments, BCI anonymizer 344 can use Empirical Mode Decomposition with Canonical Correlation Analysis (CCA) to remove movement artifacts from EEG signals. In other embodiments, the real time decomposition of brain neural signals/time series can use other or additional time-frequency approach(es); e.g., wavelets and/or other time-frequency related signal processing approaches for signal decomposition.

Once decomposed and perhaps filtered to remove artifacts, BCI anonymizer 344 can adjust properties of intrinsic mode functions, wavelets, basis functions, or other data about decomposed time series and/or decomposed signals containing extracted features. The properties can be adjusted based on importance values related to user privacy and to uses of BCI-enabled application 360 as well as signal-related aspects of the ERP component observed during calibration or other times. For example, suppose a time series or extracted features related to ERP component E2 was decomposed into functions F1, F2, . . . Fm, and functions F1 and F2 were related to privacy-sensitive information based on information-criticality metrics for the component, while functions F3 . . . Fm were not related to privacy-sensitive information. Then, BCI anonymizer 344 can reconstruct the input time series or extracted features related to ERP component using data about functions F3 . . . Fm but removing, or perhaps diminishing, data about functions F1 and F2. Other examples are possible as well.

In some embodiments, BCI anonymizer 344 can operate on features provided by feature extraction component 342 to remove privacy-sensitive information from signal features. In other embodiments, BCI anonymizer 344 can operate before signal extraction to remove privacy-sensitive information from time series and/or digital brain neural signals 330. In these embodiments, feature extraction component 342 can generate extracted features without the removed privacy-sensitive information and provide the extracted features to decoding component 346 and/or BCI-enabled application 360.

Decoding component 346 can receive anonymized extracted features as input(s) from BCI anonymizer 344 and generate application commands 350 as output. Application commands 350 can control operation of BCI-enabled application 360; e.g., include commands to move a cursor for a graphical user interface acting as BCI-enabled application 360, commands to replace a misspelled word for a word processor acting as BCI-enabled application 360. In some embodiments, decoding component 346 can be part of or related to BCI-enabled application 360; e.g., decoding component 346 can be implemented on a computing device running BCI-related application 360.

System 300 of FIG. 3 shows BCI-enabled application 360 as separate from brain-computer interface 312. For example, BCI-enabled application 360 can be a software application executing on a computing device, robotic device, or some other device. Brain-computer interface 312 can communicate anonymized extracted features and/or application commands 350 with BCI-enabled application 360 using communications that are based on a signaling protocol, such as but not limited to, a Bluetooth® protocol, a Wi-Fi® protocol, a Serial Peripheral Interface (SPI) protocol, a Universal Serial Bus (USB) protocol and/or a ZigBee® protocol. In other embodiments not shown in FIG. 3, BCI-enabled application 360 can be a component of brain-computer interface 312; e.g., an alpha wave monitoring program executed by brain-computer interface 312.

FIG. 3 shows BCI-enabled application 360 including BCI interpretation component 362 and application software 364. In embodiments where decoding component 346 is part of BCI interpretation component 362, BCI interpretation component 362 can receive anonymized extracted features from brain-computer interface 312 and generate application commands to control application software 364. In embodiments where decoding component 346 is part of brain-computer interface 312, BCI interpretation component 362 can receive application commands 350 from brain-computer interface 312, modify application commands 350 as necessary for use with application software 364, and provide received, and perhaps modified, application commands 350 to application software 364.

Application software 364 can carry out the application commands to perform application operations 370, such as but not limited to word processing operations, game-related operations, operations of a graphical user interface, operations for a command-line interface, operations to control a remotely-controllable device, such as a robotic device or other remotely-controllable device, and operations to communicate with other devices and/or persons. Many other examples of application operations 370 are possible as well.

BCI Calibration Using Information Criticality and Exposure Feasibility

A brain-computer interface, such as brain-computer interfaces 110, 160, 190, and/or 312, can be calibrated. In some embodiments, calibration information is computing using the brain-computing device, but other embodiments can use a device or service, such as but not limited to a computing device, network-based, or cloud service, connected to the brain-computing device to perform calibration and return the results to the brain-computing device.

The system can use calibration data to distinguish features, such as ERP components, of input brain neural signals and/or to determine whether features include privacy-related information.

The system can operate properly throughout the day despite fundamental changes to the inputs generated by the user; e.g., changes in electrode position, changes in sleep/waking state, changes in activity, etc. Accordingly, calibration can be performed frequently; e.g., several times a day. Calibration can be triggered periodically; i.e., at time increments, manually triggered by the user or other person, or automatically triggered. For example, calibration can be suggested or triggered when the brain-computer interface is initially powered up, or when a user indicates that they are first using the brain-computer interface.

FIG. 4 is a flowchart of calibration method 400. Method 400 can begin at block 410, where calibration start input is received at one or more devices calibrating a system for operating a brain-computing interface, such as but not limited to brain-computer interface 110, 160, 190, or 312. In some embodiments, the brain-computer interface can perform part or all of method 400, while in other embodiments, one or more other devices, such as computing devices, can perform part or all of calibration method 400.

At block 410, the start calibration input can be periodic or otherwise time based; e.g., calibration process 400 can be performed every 30 minutes, every two hours, or every day. The start calibration input can be a manual input; e.g., a button is pressed or other operation performed by a user or other entity to initiate calibration process 400.

Calibration can be performed partially or completely automatically. As an example, calibration can be performed upon a manual input to power up the brain-computer interface; e.g., the power button is pressed or otherwise activated for the brain-computer interface. In particular, a “second power up” input can trigger calibration; that is, input to power up a brain-computer interface or an associated computing device performing by itself will not trigger calibration, but input for powering up the latter-powered-up of the brain-computer interface and associated computing device so that both devices are powered up can trigger calibration. Also, identification of a new user of the brain-computer interface can trigger calibration for the new user.

At block 420, a decision is made to perform the remainder of calibration method 400. If calibration is to be performed, calibration method 400 can proceed to block 430. Otherwise, calibration is not to be performed and calibration method 400 can proceed to block 492 to end.

At block 430, a determination is made to which feature F1 to calibrate. Example features include ERP components, including but not limited to the N100, P300, N400, P600 and ERN ERP components. Other features and/or ERP components can be calibrated as well.

At block 440, one or more stimuli related to feature F1 are provided to a user calibrating the brain-computer interface. The one or more stimuli related to FI can include visual stimuli, auditory stimuli, and/or touch-oriented stimuli. In some embodiments, the stimuli intended to obtain certain feature-related responses, such as target-stimulus reactions or non-target-stimulus reactions. In other embodiments, multiple trials of the stimuli are provided to the user, so that feature-related data from multiple trials can be averaged or otherwise combined to determine feature-related data for feature F1, FRD(F1).

At block 450, the brain-computer interface can receive brain neural signals related to the stimuli presented at block 440. The brain neural signals can be acquired, conditioned, digitized, and feature-related data for F1, FRD(F1), can be extracted from the brain neural signals. For example, the brain-computer interface can use a signal conditioning component and at least a feature extraction component of a digital signal processing component to extract feature-related data for F1 as discussed above in the context of FIG. 3.

At block 460, feature-related data for F1, FRD(F1), can be attempted to be certified. In this context, FRD(F1) can be certified for suitability for calibration of feature F1. FRD(F1) may not be certified if FRD(F1) is: unrelated to feature F1, includes too much noise for calibration, is too weak or too strong for calibration, or for other reasons.

At block 470, a determination can be made whether FRD(F1) is certified for calibration of feature F1. If FRD(F1) is not certified for calibration for feature F1, additional feature-related data can be obtained by having calibration process 400 proceed to block 440. Otherwise, FRD(F1) is certified for calibration, and calibration process 400 can proceed to block 480.

At block 480, FRD(F1) can be certified as signal-related data for calibration of feature F1. Also, additional data can be obtained related to privacy-sensitive information related to feature F1, such as but not limited to, information-criticality metric data for σ(F1) and/or exposure feasibility data for η(F1).

A relative reduction in entropy, defined based on relative reduction of Shannon's entropy with respect to a random guess of private information, can be used to quantify an amount of extracted privacy-sensitive information. Equation (1) can be used to quantify the relative reduction in entropy.

$$r ( clf ) := 100 \frac{H ( X \text{\textbar} {a}^{( rand )} ) - H ( X \text{\textbar} {a}^{( clf )} )}{H ( X \text{\textbar} {a}^{( rand )} )} = 100 - \frac{100 H ( X \text{\textbar} {a}^{( clf )} )}{{log}_{2} ( K )} \qquad ( 1 )$$

where r(clf) denotes the reduction in entropy, clf is the classifier used, H(X|a^{(rand)}) is the Shannon entropy of a random guess, H(X|a^{(clf)}) is the Shannon entropy with classifier clf, and K the number of possible answers to the private information.

Equation (1) defines the reduction in entropy as a function with respect to a chosen classifier clf. In other models of extracted privacy-sensitive information, the reduction in entropy can be a function of a chosen ERP component; e.g., feature F1, the user's level of facilitating data extraction, and/or the user's awareness of presented stimuli.

Another quantification of amounts of privacy-sensitive information is based on an assumption that not all privacy-sensitive information is equally important to a subject. To quantify an importance of information to an individual, an information-criticality metric for feature F1, σ(F1), can be used, where σ(F1) ε[0, 1], and with σ(F1)=0 indicating F1 relates to most-important information for the individual and σ(1)=1 indicating F1 relates to information without privacy-related importance to the individual.

The relative reduction in entropy can be defined as a function of a chosen ERP component and the information-criticality metric. This definition of relative reduction of entropy can be referred to as the exposure feasibility, η, as quantified in Equation (2), which can quantify the usefulness of a chosen ERP component ERP_{comp}; e.g., feature F1, in extracting the set of private information, S_{PI}.

$$η ( {ERP}_{comp} ) := {\sum }_{i = 1}^{\vert {S}_{P} \vert} {r ( {ERP}_{comp} )}_{i} {σ}_{i} \qquad ( 2 )$$

where ERP_{comp} is the chosen ERP component such as feature F1, S_{PI} is the set of private information, i is an index selecting a particular portion of information in S_{PI}, r(ERP_{comp})ᵢ is a reduction in entropy for the chosen ERP component with respect to the particular portion of information, and σᵢ is the information-criticality metric with respect to the particular portion of information. Then, given two ERP components, C1 and C2, η(C1)<η(C2) indicates that C1 is a more useful ERP component for extracting private data than C2.

The brain-computer interface generally, and a BCI anonymizer of the brain-computer interface specifically, can be calibrated for a user of a brain-computer interface. During block 480, attacks on privacy-sensitive information can be simulated—the user can inadvertently facilitate extraction of privacy-sensitive information by following simulated malicious applications' instructions. Also, information about feature F1 can be presented to the user; e.g., information about how feature F1 relates to user privacy, and questions asked related to the user's opinion on the information-criticality of F1, σ(F1). From data about the simulated attacks, data from previous blocks of method 400, and data related to the user's opinion on σ(F1), information for determining information-criticality of feature F1, σ(F1), and/or exposure-feasibility for feature F1, η(F1) can be obtained.

At block 490, a determination is made as to whether there are more features or other signals to calibrate. If there are more channel states or other signals to calibrate, calibration method 400 can proceed to block 430. Otherwise, there are no more features or other signals to calibrate and calibration method 400 can proceed to block 492 to end.

In some embodiments, the system can protect the privacy of users and protect communications from interception. To protect privacy, communications between the brain-computer interface and associated device(s) used to calibrate the brain-computer interface can be encrypted or otherwise secured. The brain-computer interface and/or associated device(s) can be protected by passwords or biometrics from unauthorized use. In particular embodiments, calibration method 400 can be used to provide biometric information to protect the brain-computer interface. For example, the user can be requested to perform a calibration session to generate current input channel signal data. The current input channel signal data can be compared to previously-stored input channel signal data. If the current input channel signal data at least approximately matches the previously-stored input channel signal data, then the brain-computer interface can determine that the current user is the previous user, and assume the brain-computer interface is being used by the correct, and current, user.

Point-to-point links, e.g., a Bluetooth® paired link, a wired communication link, can be used to reduce (inadvertent) interception of system communications. For more public systems, such as systems using Wi-Fi® or Wireless Wide Area Network (WWAN) communications, secure links and networks can be used to protect privacy and interception. The system can also use communication techniques, such as code sharing and time-slot allocation, that protect against inadvertent and/or intentional interception of communications. Many other techniques to protect user security and communication interception can be used by the system as well.

Example Computing Network

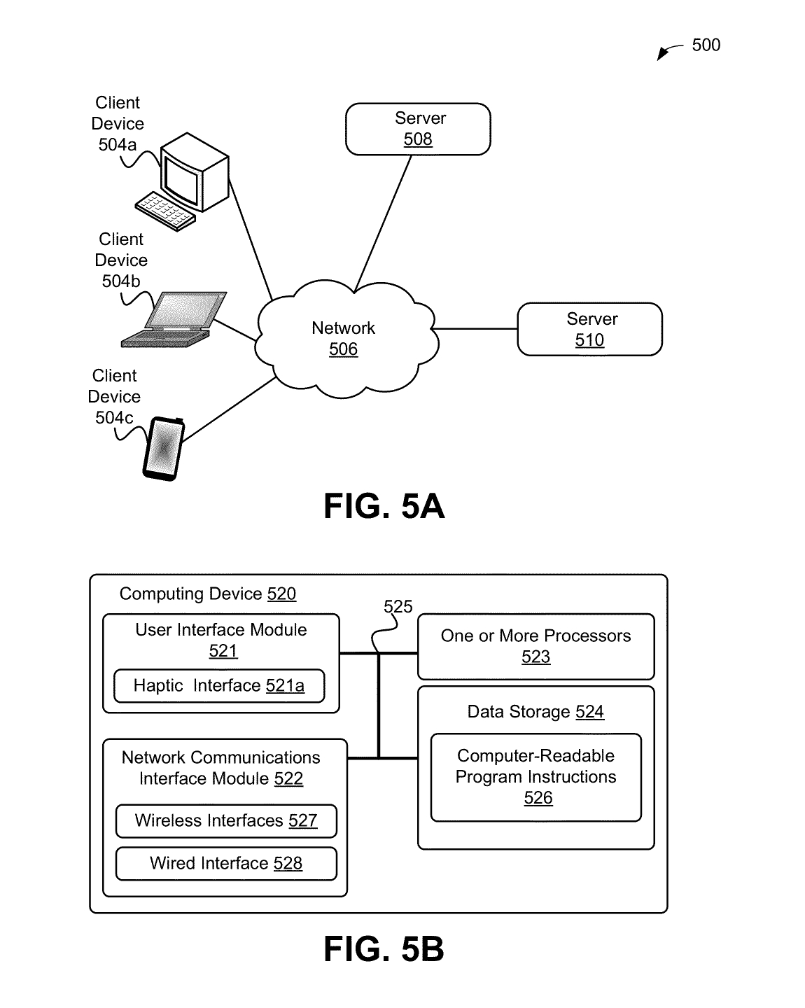

FIG. 5A is a block diagram of example computing network 500 in accordance with an example embodiment. In FIG. 5A, servers 508 and 510 are configured to communicate, via a network 506, with client devices 504a, 504b, and 504c. As shown in FIG. 5A, client devices can include a personal computer 504a, a laptop computer 504b, and a smart-phone 504c. More generally, client devices 504a-504c (or any additional client devices) can be any sort of computing device, such as a workstation, network terminal, desktop computer, laptop computer, wireless communication device (e.g., a cell phone or smart phone), and so on. In some embodiments, some or all of client devices 504a-504c can include or be associated with a brain-computer interface; e.g., one or more of brain-computer interfaces 120, 160, 190, and/or 312.

The network 506 can correspond to a local area network, a wide area network, a corporate intranet, the public Internet, combinations thereof, or any other type of network(s) configured to provide communication between networked computing devices. In some embodiments, part or all of the communication between networked computing devices can be secured.

Servers 508 and 510 can share content and/or provide content to client devices 504a-504c. As shown in FIG. 5A, servers 508 and 510 are not physically at the same location. Alternatively, servers 508 and 510 can be co-located, and/or can be accessible via a network separate from network 506. Although FIG. 5A shows three client devices and two servers, network 506 can service more or fewer than three client devices and/or more or fewer than two servers. In some embodiments, servers 508, 510 can perform some or all of the herein-described methods; e.g., method 400 and/or method 600.

Example Computing Device

FIG. 5B is a block diagram of an example computing device 520 including user interface module 521, network-communication interface module 522, one or more processors 523, and data storage 524, in accordance with an embodiment.

In particular, computing device 520 shown in FIG. 5A can be configured to perform one or more functions of systems 100, 150, 180a, 180b, 300, brain-computer interfaces 120, 160, 190, 312, computing devices 120, 170, signal acquisition component 320, digital signal processing component 340, BCI-enabled application 122, 172, 360, client devices 504a-504c, network 506, and/or servers 508, 510. Computing device 520 may include a user interface module 521, a network-communication interface module 522, one or more processors 523, and data storage 524, all of which may be linked together via a system bus, network, or other connection mechanism 525.

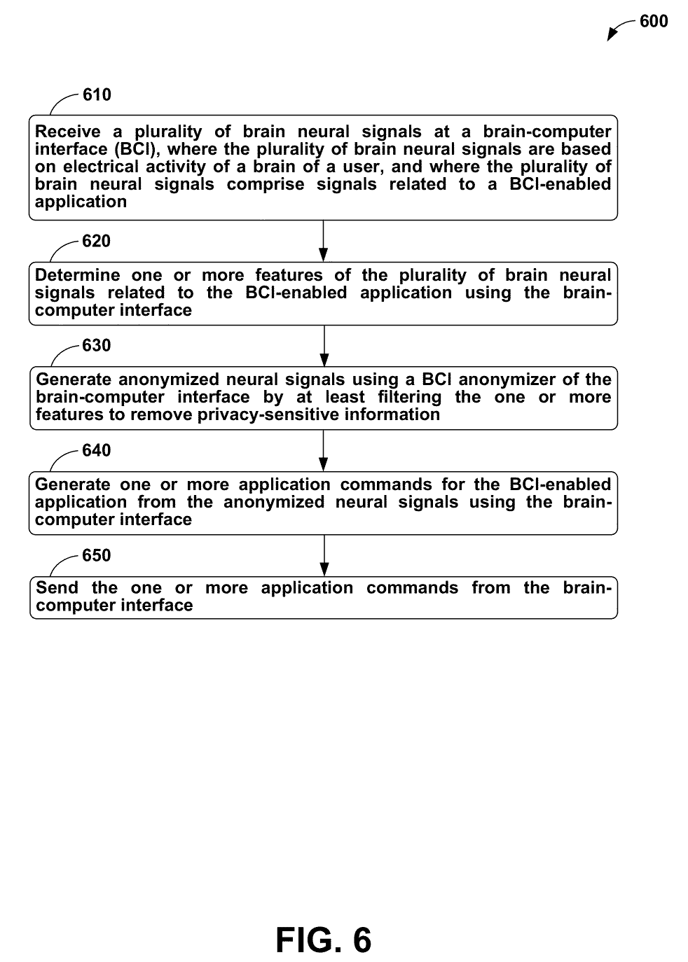

Computing device 520 can be a desktop computer, laptop or notebook computer, personal data assistant (PDA), mobile phone, embedded processor, touch-enabled device, or any similar device that is equipped with at least one processing unit capable of executing machine-language instructions that implement at least part of the herein-described techniques and methods, including but not limited to method 400 described with respect to FIG. 4 and/or method 600 described with respect to FIG. 6.

User interface 521 can receive input and/or provide output, perhaps to a user. User interface 521 can be configured to send and/or receive data to and/or from user input from input device(s), such as a keyboard, a keypad, a touch screen, a computer mouse, a track ball, a joystick, and/or other similar devices configured to receive input from a user of the computing device 520. In some embodiments, input devices can include BCI-related devices, such as, but not limited to, brain-computer interfaces 110, 160, 190, and/or 312.

User interface 521 can be configured to provide output to output display devices, such as one or more cathode ray tubes (CRTs), liquid crystal displays (LCDs), light emitting diodes (LEDs), displays using digital light processing (DLP) technology, printers, light bulbs, and/or other similar devices capable of displaying graphical, textual, and/or numerical information to a user of computing device 520. User interface module 521 can also be configured to generate audible output(s), such as a speaker, speaker jack, audio output port, audio output device, earphones, and/or other similar devices configured to convey sound and/or audible information to a user of computing device 520. As shown in FIG. 5B, user interface can be configured with haptic interface 521a that can receive haptic-related inputs and/or provide haptic outputs such as tactile feedback, vibrations, forces, motions, and/or other touch-related outputs.

Network-communication interface module 522 can be configured to send and receive data over wireless interface 527 and/or wired interface 528 via a network, such as network 506. Wireless interface 527 if present, can utilize an air interface, such as a Bluetooth®, Wi-Fi®, ZigBee®, and/or WiMAX™ interface to a data network, such as a wide area network (WAN), a local area network (LAN), one or more public data networks (e.g., the Internet), one or more private data networks, or any combination of public and private data networks. Wired interface(s) 528, if present, can comprise a wire, cable, fiber-optic link and/or similar physical connection(s) to a data network, such as a WAN, LAN, one or more public data networks, one or more private data networks, or any combination of such networks.

In some embodiments, network-communication interface module 522 can be configured to provide reliable, secured, and/or authenticated communications. For each communication described herein, information for ensuring reliable communications (i.e., guaranteed message delivery) can be provided, perhaps as part of a message header and/or footer (e.g., packet/message sequencing information, encapsulation header(s) and/or footer(s), size/time information, and transmission verification information such as CRC and/or parity check values). Communications can be made secure (e.g., be encoded or encrypted) and/or decrypted/decoded using one or more cryptographic protocols and/or algorithms, such as, but not limited to, DES, AES, RSA, Diffie-Hellman, and/or DSA. Other cryptographic protocols and/or algorithms can be used as well as or in addition to those listed herein to secure (and then decrypt/decode) communications.

Processor(s) 523 can include one or more central processing units, computer processors, mobile processors, digital signal processors (DSPs), microprocessors, computer chips, and/or other processing units configured to execute machine-language instructions and process data. Processor(s) 523 can be configured to execute computer-readable program instructions 526 that are contained in data storage 524 and/or other instructions as described herein.

Data storage 524 can include one or more physical and/or non-transitory storage devices, such as read-only memory (ROM), random access memory (RAM), removable-disk-drive memory, hard-disk memory, magnetic-tape memory, flash memory, and/or other storage devices. Data storage 524 can include one or more physical and/or non-transitory storage devices with at least enough combined storage capacity to contain computer-readable program instructions 526 and any associated/related data structures.

Computer-readable program instructions 526 and any data structures contained in data storage 526 include computer-readable program instructions executable by processor(s) 523 and any storage required, respectively, to perform at least part of herein-described methods, including, but not limited to, method 400 described with respect to FIG. 4 and/or method 600 described with respect to FIG. 6.

Example Methods of Operation

FIG. 6 is a flow chart of an example method 600. Method 600 can be carried out by a brain-computer interface, such as brain-computer interfaces 110, 160, 190, and/or 312, such as discussed above in the context of at least FIGS. 1A, 1B, 1C, and 3.

Method 600 can begin at block 610, where a brain-computer interface can receive a plurality of brain neural signals, such as discussed above in the context of at least FIGS. 1A, 1B, 1C, and 3. The plurality of brain neural signals can be based on electrical activity of a brain of a user and can include signals related to a BCI-enabled application.

At block 620, the brain-computer interface can determine one or more features of the plurality of brain neural signals related to the BCI-enabled application, such as discussed above in the context of at least FIG. 3.

In some embodiments, the one or more features can include one or more ERP components of the plurality of brain neural signals, such as discussed above in the context of at least FIG. 3.

At block 630, a BCI anonymizer of the brain-computer interface can generate anonymized neural signals by at least filtering the one or more features to remove privacy-sensitive information, such as discussed above in the context of at least FIG. 3.

In some embodiments, the BCI anonymizer generating anonymized neural signals includes the BCI anonymizer generating anonymized neural signals from the one or more ERP components. In particular embodiments, the BCI anonymizer generating anonymized neural signals from the one or more ERP components includes the BCI anonymizer decomposing the one or more ERP components into a plurality of functions; modifying at least one function of the plurality of functions to remove the privacy-sensitive information from the plurality of functions; and generating the anonymized neural signals using the modified plurality of functions.

In more particular embodiments, the BCI anonymizer decomposing the one or more ERP components into a plurality of functions includes the BCI anonymizer performing real-time decomposition of the ERP components into the plurality of functions using a time-frequency signal processing algorithm. In still more particular embodiments, the time-frequency signal processing algorithm can include at least one algorithm selected from the group consisting of an algorithm utilizing wavelets and an algorithm utilizing empirical mode decomposition.

In other embodiments, the BCI anonymizer generating anonymized neural signals from the one or more ERP components includes the BCI anonymizer determining an information-criticality metric for at least one feature of the one or more features and filtering the one or more features to remove privacy-sensitive information based on the information-criticality metric for the at least one feature.

In particular of the other embodiments, the BCI anonymizer filtering the one or more features to remove privacy-sensitive information based on the information-criticality metric for the at least one feature includes the BCI anonymizer determining a relative reduction in entropy for the at least one feature based on the information-criticality metric for the at least one feature.

At block 640, the brain-computer interface can generate one or more application commands for the BCI-enabled application from the anonymized neural signals, such as discussed above in the context of at least FIG. 3.

At block 650, the brain-computer interface can send the one or more application commands, such as discussed above in the context of at least FIG. 3.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

The above description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure. The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The above detailed description describes various features and functions of the disclosed systems, devices, and methods with reference to the accompanying figures. In the figures, similar symbols typically identify similar components, unless context dictates otherwise. The illustrative embodiments described in the detailed description, figures, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the ladder diagrams, scenarios, and flow charts in the figures and as discussed herein, each block and/or communication may represent a processing of information and/or a transmission of information in accordance with example embodiments. Alternative embodiments are included within the scope of these example embodiments. In these alternative embodiments, for example, functions described as blocks, transmissions, communications, requests, responses, and/or messages may be executed out of order from that shown or discussed, including substantially concurrent or in reverse order, depending on the functionality involved. Further, more or fewer blocks and/or functions may be used with any of the ladder diagrams, scenarios, and flow charts discussed herein, and these ladder diagrams, scenarios, and flow charts may be combined with one another, in part or in whole.

A block that represents a processing of information may correspond to circuitry that can be configured to perform the specific logical functions of a herein-described method or technique. Alternatively or additionally, a block that represents a processing of information may correspond to a module, a segment, or a portion of program code (including related data). The program code may include one or more instructions executable by a processor for implementing specific logical functions or actions in the method or technique. The program code and/or related data may be stored on any type of computer readable medium such as a storage device including a disk or hard drive or other storage medium.

The computer readable medium may also include non-transitory computer readable media such as computer-readable media that stores data for short periods of time like register memory, processor cache, and random access memory (RAM). The computer readable media may also include non-transitory computer readable media that stores program code and/or data for longer periods of time, such as secondary or persistent long term storage, like read only memory (ROM), optical or magnetic disks, compact-disc read only memory (CD-ROM), for example. The computer readable media may also be any other volatile or non-volatile storage systems. A computer readable medium may be considered a computer readable storage medium, for example, or a tangible storage device.

Moreover, a block that represents one or more information transmissions may correspond to information transmissions between software and/or hardware modules in the same physical device. However, other information transmissions may be between software modules and/or hardware modules in different physical devices.

Numerous modifications and variations of the present disclosure are possible in light of the above teachings.

What is claimed:

1. A method, comprising:
receiving a plurality of brain neural signals at a brain-computer interface (BCI), wherein the plurality of brain neural signals are based on electrical activity of a brain of a user, and wherein the plurality of brain neural signals comprise signals related to a BCI-enabled application;
determining one or more features of the plurality of brain neural signals related to the BCI-enabled application using the brain-computer interface;
generating anonymized neural signals using a BCI anonymizer of the brain-computer interface by at least filtering the one or more features to remove privacy-sensitive information;
generating one or more application commands for the BCI-enabled application from the anonymized neural signals using the brain-computer interface; and
sending the one or more application commands from the brain-computer interface.

2. The method of claim 1, wherein the one or more features comprise one or more event-related-potential (ERP) components of the plurality of brain neural signals.

3. The method of claim 2, wherein generating anonymized neural signals comprises generating anonymized neural signals from the one or more ERP components using the BCI anonymizer.

4. The method of claim 3, wherein generating anonymized neural signals from the one or more ERP components using the BCI anonymizer comprises:
decomposing the one or more ERP components into a plurality of functions;
modifying at least one function of the plurality of functions to remove the privacy-sensitive information from the plurality of functions; and
generating the anonymized neural signals using the modified plurality of functions.

5. The method of claim 4, wherein decomposing the one or more ERP components into the plurality of functions comprises performing real-time decomposition of the ERP components into the plurality of functions using a time-frequency signal processing algorithm.

6. The method of claim 5, wherein the time-frequency signal processing algorithm is at least one algorithm selected from the group consisting of an algorithm utilizing wavelets and an algorithm utilizing empirical mode decomposition.

7. The method of claim 3, generating anonymized neural signals from the one or more ERP components using the BCI anonymizer comprises:
determining an information-criticality metric for at least one feature of the one or more features; and
filtering the one or more features to remove privacy-sensitive information based on the information-criticality metric for the at least one feature.

8. The method of claim 7, wherein filtering the one or more features to remove privacy-sensitive information based on the information-criticality metric for the at least one feature comprises determining a relative reduction in entropy for the at least one feature based on the information-criticality metric for the at least one feature.

9. A brain-computer interface (BCI), comprising:
a signal acquisition component, configured to receive a plurality of brain neural signals based on electrical activity of a brain of a user, and wherein the plurality of brain neural signals comprise signals related to a BCI-enabled application; and
a signal processing component, comprising:
a feature extraction component, configured to determine one or more features of the plurality of brain neural signals related to the BCI-enabled application,
a BCI anonymizer, configured to generate anonymized neural signals by at least filtering the one or more features to remove privacy-sensitive information, and
a decoding component, configured to generate one or more application commands for the BCI-enabled application from the anonymized neural signals.

10. The brain-computer interface of claim 9, wherein the one or more features comprise one or more event-related-potential (ERP) components of the plurality of brain neural signals.

11. The brain-computer interface of claim 10, wherein the BCI anonymizer is configured to generate the anonymized neural signals from the one or more ERP components.

12. The brain-computer interface of claim 11, wherein the BCI anonymizer is configured to generate the anonymized neural signals from the one or more ERP components by at least:
decomposing the one or more ERP components into a plurality of functions;
modifying at least one function of the plurality of functions to remove the privacy-sensitive information from the plurality of functions; and
generating the anonymized neural signals using the modified plurality of functions.

13. The brain-computer interface of claim 12, wherein decomposing the one or more ERP components into the plurality of functions comprises performing real-time decomposition of the ERP components into the plurality of functions using a time-frequency signal processing algorithm.

14. The brain-computer interface of claim 13, wherein the time-frequency signal processing algorithm comprises at least one algorithm selected from the group consisting of an algorithm utilizing wavelets and an algorithm utilizing empirical mode decomposition.

15. The brain-computer interface of claim 11, wherein the BCI anonymizer is configured to generate the anonymized neural signals from the one or more ERP components by at least:
determining an information-criticality metric for at least one feature of the one or more features; and
filtering the one or more features to remove privacy-sensitive information based on the information-criticality metric for the at least one feature.

16. The brain-computer interface of claim 15, wherein filtering the one or more features to remove privacy-sensitive information based on the information-criticality metric for the at least one feature comprises determining a relative reduction in entropy for the at least one feature based on the information-criticality metric for the at least one feature.

17. An article of manufacture comprising a non-transitory tangible computer readable medium configured to store at least executable instructions, wherein the executable instructions, when executed by a processor of a brain-computer interface (BCI), cause the brain-computer interface to perform functions comprising:
determining one or more features of a plurality of brain neural signals related to a BCI-enabled application;
generating anonymized neural signals by at least filtering the one or more features to remove privacy-sensitive information;
generating one or more application commands for the BCI-enabled application from the anonymized neural signals; and
sending the one or more application commands from the brain-computer interface.

18. The article of manufacture of claim 17, wherein the one or more features comprise one or more event-related-potential (ERP) components, and wherein generating the anonymized neural signals by at least filtering the one or more features comprises:
decomposing the one or more ERP components into a plurality of functions;
modifying at least one function of the plurality of functions to remove the privacy-sensitive information from the plurality of functions; and
generating the anonymized neural signals using the modified plurality of functions.

19. The article of manufacture of claim 18, wherein decomposing the one or more ERP components into the plurality of functions comprises performing real-time decomposition of the ERP components into the plurality of functions using a time-frequency signal processing algorithm.

20. The article of manufacture of claim 19, wherein the time-frequency signal processing algorithm comprises at least one algorithm selected from the group consisting of an algorithm utilizing wavelets and an algorithm utilizing empirical mode decomposition.
